(19) United States
(12) Patent Application Publication
(10) Pub. No.: US 2025/0266174 A1
(43) Pub. Date: Aug. 21, 2025

(54) SYSTEM AND METHOD FOR AUTOMATED DATA EXTRACTION AND ANALYSIS OF FDA 505(B)(2) APPLICATIONS

(71) Applicant: BUCHANAN, INGERSOLL & ROONEY PC, Alexandria, VA (US)

(72) Inventors: Matthew DIPPOLD, Wexford, PA (US); Christopher GADSBY, McKees Rocks, PA (US)

(73) Assignee: BUCHANAN, INGERSOLL & ROONEY PC, Alexandria, VA (US)

(21) Appl. No.: 19/054,645

(22) Filed: Feb. 14, 2025

Related U.S. Application Data
(60) Provisional application No. 63/554,291, filed on Feb. 16, 2024.

(51) Int. Cl. G16H 70/40; G06F 40/279; G06F 40/40

(52) U.S. Cl. CPC G16H 70/40; G06F 40/279; G06F 40/40

(57) ABSTRACT

Systems and methods are provided for automatically generating reports of new drug applications, including memory storing program instructions and at least one processor programmed or configured to retrieve plural data files from a server associated with the Food and Drug Administration (FDA), wherein the plural data files include data associated with new drug applications; determine that at least one data file includes data for an approved 505(b)(2) drug application; generate a list of data files including a subset of plural data files, wherein each data file in the subset of plural data files represents a new drug application; input the subset of plural data files including data for approved 505(b)(2) drug applications into at least one natural language processing (NLP) model; and generate, with the at least one NLP model, a text summary of each data file of the subset of plural data files.

### CROSS-REFERENCE

This U.S. Patent Application is related to and claims priority to U.S. Provisional Application No. 63/554,291, filed on Feb. 16, 2024, the entire contents of which are incorporated herein by reference.

### BACKGROUND

This disclosure relates generally to automated data extraction from data files for data analysis and processing, and more particularly to systems and methods for automated data extraction and analysis of United States Food and Drug Administration (FDA) 505(b)(2) applications to generate summaries and/or reports using automated processes and/or natural language processing.

Typically, new drugs in the United States may be approved by the FDA through one or more application pathways. At least one application pathway, a New Drug Application (NDA) filed under section 505(b)(2) of the Federal Food, Drug, and Cosmetic Act, may be an option in some instances. Entities may submit an NDA under 505(b)(2) and may submit data associated with the NDA in order to fulfill requirements for the application pathway to achieve approval. Data associated with new drug applications (e.g., an NDA filed under 505(b)(2)) submitted through the one or more application pathways may be collected via computer input and/or electronic application methods. The FDA may store the data associated with new drug applications on one or more servers, where the data may be accessible to the public via the Internet.

In some instances, the FDA may store the data associated with new drug applications on one or more servers in a Portable Document Format (PDF) or a text file (e.g., *.txt) format, such that data associated with new drug applications for each individual drug application may be stored as single PDF files and/or single data files of another type. Such a storage mechanism may be simple to achieve, however this storage layout and/or file organization may be difficult to navigate. In order to view a large amount of the data associated with new drug applications stored on the FDA server, a client device (e.g., a user using a client device) would need to download each data file individually onto the client device and manually search through each data file to search, collect, and analyze data that may be useful. In order to get a wholistic picture of the data associated with new drug applications, a user would need to manually analyze all of the PDF files to compile a complete analysis. Each of these techniques would require vast amounts of time and resources for a user and would also require use of a client device having a large amount of computing power and storage in order to download, review, store, and organize all available data associated with new drug applications.

In some instances, the data associated with new drug applications may be inaccessible, difficult to locate, or cumbersome for users to search through text or image data in PDF files or other data files. Sometimes, data files stored on the FDA server may be extremely large data files, requiring large amounts of storage space and may be impractical for a user to search through manually in order to find a small amount of information or to generate a complete analysis of new drug applications. Additionally, the data associated with new drug applications may be stored in PDF files or other data files in a text and/or image format, further complicating a data analysis process with large datasets and varying data types. As NDAs are submitted, new data is added to the data associated with new drug applications stored on the FDA server without alerting users or without effectively updating any analyses that may have been previously completed.

A problem of downloading, viewing, analyzing, and displaying data associated with new drug applications is exacerbated by a lack of tools, systems, and/or techniques to identify the data, compile the data, and analyze the data such that large amounts of data associated with new drug applications can be summarized and displayed efficiently for users to comprehend. Another problem may include difficulty in identifying new drug applications, identifying delimiters for new drug applications, and analyzing the data contained therein. Further problems may include difficulty identifying specific data (e.g., a date of approval for an NDA) contained within large data files of data associated with new drug applications (e.g., within a single data file) and organizing or grouping desired data sets based on identifying the specific data within the large data files.

### SUMMARY

Accordingly, provided are systems and methods for automated data extraction and analysis of FDA 505(b)(2) applications to generate summaries and/or reports using automated computer processing and/or natural language processing.

Embodiments may relate tjo a system for automatically generating reports of new drug applications. The system may include memory storing program instructions and at least one processor configured to execute the program instructions. When the at least one processor loads and executes the program instructions, the at least one processor may be programmed or configured to retrieve plural data files from a server associated with the FDA. The plural data files may include data associated with new drug applications. The at least one processor may be programmed or configured to determine that at least one data file includes data for an approved 505(b)(2) drug application. The at least one processor may be programmed or configured to generate a list of data files including a subset of plural data files. Each data file in the subset of plural data files may represent a new drug application or a biologics license application (BLA). The at least one processor may be programmed or configured to input the subset of plural data files including data for approved 505(b)(2) drug applications into at least one natural language processing (NLP) model. The at least one processor may be programmed or configured to generate, with the at least one NLP model, a text summary of each data file of the subset of plural data files.

Embodiments may relate to a method for automatically generating reports for new drug applications. The method may include receiving plural data files from a server. The plural data files may include data representing new drug applications submitted to a government agency. The method may include extracting the data representing new drug applications from the plural data files to generate extracted text data. The method may include analyzing the extracted text data using an NLP model. The method may include determining that at least one data file includes data for an approved 505(b)(2) drug application based on analyzing the extracted text data using the NLP model. The method may include generating a data table based on analyzing the extracted text data. The data table may include one or more data files of the plural data files that were determined to include data for an approved 505(b)(2) drug application. Each data file of the one or more data files may be associated with a flag in the data table. The method may include generating, with the at least one NLP model, a summary of the data for an approved 505(b)(2) drug application in each data file of the one or more data files in the data table.

Embodiments may relate to a computer program product for automatically generating reports for new drug applications. The computer-program product may include at least one non-transitory computer-readable medium storing instructions that, when loaded and executed by at least one processor, cause the at least one processor to retrieve plural data files from a server associated with the FDA. The plural data files may include data representing new drug applications. The instructions may cause the at least one processor to determine that at least one data file includes data for an approved 505(b)(2) drug application. The instructions may cause the at least one processor to generate a list of data files including a subset of plural data files. Each data file in the subset of plural data files may represent a new drug application or a BLA. The instructions may cause the at least one processor to input the subset of plural data files including data for approved 505(b)(2) drug applications into at least one natural language processing (NLP) model. The instructions may cause the at least one processor to generate, with the at least one NLP model, a text summary of each data file of the subset of plural data files.

These and other features and characteristics of the present disclosure, as well as the methods of operation and functions of the related elements of structures and the combination of parts and economies of manufacture, will become more apparent upon consideration of the following description and the appended claims with reference to the accompanying drawings and appendix, all of which form a part of this specification, wherein like reference numerals designate corresponding parts in the various figures. It is to be expressly understood, however, that the drawings and appendix are for the purpose of illustration and description only and are not intended as a definition of the limits of the disclosed subject matter.

### BRIEF DESCRIPTION OF THE DRAWINGS AND APPENDIX

Additional advantages and details are explained in greater detail below with reference to the embodiments that are illustrated in the accompanying schematic figures, in which:

FIG. 1 is a schematic diagram of a system configuration for automated data extraction and analysis of FDA 505(b)(2) applications to generate summaries and/or reports using automated computer processing and/or natural language processing according to some embodiments;

FIG. 2 is a flow diagram of a method for automated data extraction and analysis of FDA 505(b)(2) applications to generate summaries and/or reports using automated computer processing and/or natural language processing according to some embodiments;

FIG. 3 is a schematic diagram of an exemplary environment in which systems, methods, and/or computer program products, described herein, may be implemented according to some embodiments;

FIG. 4 is a schematic diagram of example components of one or more systems and/or devices of FIG. 1 and/or FIG. 3 according to some embodiments; and

Appendix includes additional details regarding systems and methods for automated data extraction and analysis of FDA 505(b)(2) applications to generate summaries and/or reports using automated computer processing and/or natural language processing according to some embodiments.

### DETAILED DESCRIPTION

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the terms “client” and “client device” may refer to one or more client-side devices or systems used to initiate or facilitate a network connection. As an example, a “client device” may refer to one or more computing devices used by a user, one or more personal computers used by a user, one or more mobile devices used by a user, and/or the like. In some non-limiting embodiments, a client device may be an electronic device configured to communicate with one or more networks. For example, a client device may include one or more computers, portable computers, laptop computers, tablet computers, mobile devices, cellular phones, wearable devices (e.g., watches, glasses, lenses, clothing, and/or the like), PDAs, and/or the like. Moreover, a “client” may also refer to an entity (e.g., a user, a corporation, and/or the like) that owns, utilizes, and/or operates a client device.

As used herein, the term “server” may refer to or include one or more computing devices that are operated by or facilitate communication and processing for multiple parties (e.g., clients, client devices, users, and/or the like) in a network environment, such as the Internet, although it will be appreciated that communication may be facilitated over one or more public or private network environments and that various other arrangements are possible. Further, multiple computing devices (e.g., servers, mobile devices, etc.) directly or indirectly communicating in the network environment may constitute a “system.” Reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Embodiments may extract and analyze data associated with new drug applications stored on one or more servers associated with the FDA. Embodiments may extract data from one or more data files, such as data stored in a PDF file or a text file (or other file types) format. Embodiments may extract the data such that data associated with new drug applications for each individual drug application may be identified, collected, and/or stored together in a database and/or spreadsheet format. Such a storage mechanism may allow for improved data organization and may facilitate efficient organization, analysis, and/or display of the extracted data using a processor and/or a computing device.

Embodiments may store the extracted data on a single server or on distributed servers such that client devices (e.g., users using client devices) may view and access the extracted data without needing to download each data file individually onto the client device and manually search through each data file to collect, find, and analyze data that may be useful. Embodiments may generate trends (e.g., a visual display), reports, summaries, and/or other forms of data visualization objects based on the extracted data such that client devices may access the trends, reports, summaries and/or other forms of data visualization objects for displaying on a display device. Embodiments provide for efficient analysis of data associated with new drug applications, where analyses may be accessible to one or more client devices to provide for efficient display of data visualization objects. Embodiments may reduce an amount of resources required for a user to view and/or analyze data associated with new drug applications and eliminate a need for a client device to have a large amount of computing power to manage, store, and manipulate any available data associated with new drug applications in multiple data files.

Embodiments allow for the data associated with new drug applications to be accessible by multiple client devices and easy to locate and/or filter for the client devices. Embodiments may allow multiple client devices to navigate through large amounts of data quickly and easily without encapsulating the data associated with new drug applications in individual data files. Embodiments may extract data from data files in specific data formats, such that the data associated with new drug applications may be structured to work with different applications and different display visualization objects.

Embodiments may provide for querying, extracting, storing, querying, and analyzing large datasets, particularly where the data is unorganized and stored in individual data files. Embodiments may also allow for automatic tracking and discovery of new drug application applications as new data is added to a server associated with the FDA, such that data extracted and/or analyzed by some embodiments may be effectively updated in real time so that analyses may be kept current without requiring user action with a client device or computing device.

Embodiments may allow for efficient querying, downloading, viewing, analyzing, and displaying of data associated with new drug applications such that embodiments may identify the data, compile the data, and analyze the data for one or more client devices to access, view, and display data associated with new drug applications for summarization and display to reduce time and resources that would typically be required to search through and analyze accessible data associated with new drug applications in whatever format the data is provided on the FDA server.

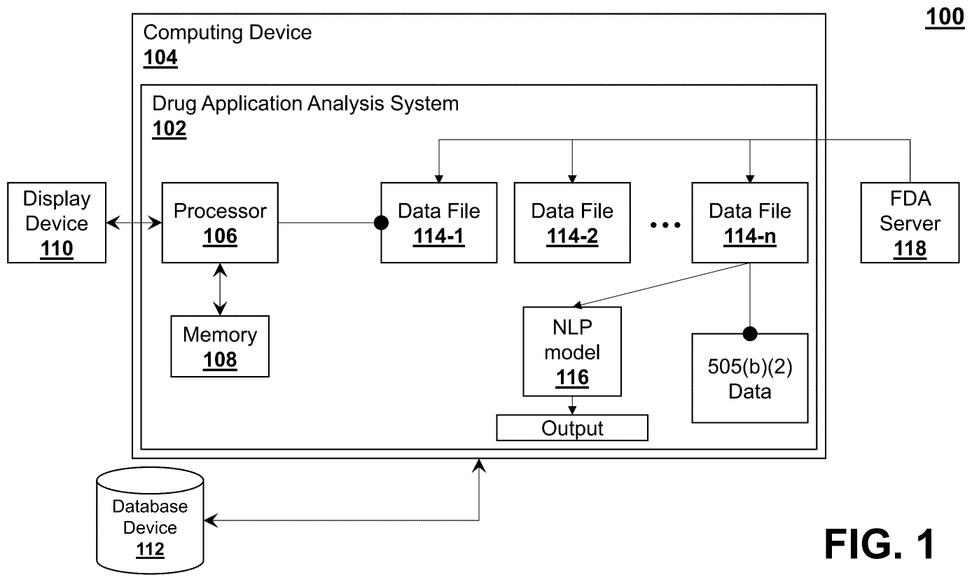

FIG. 1 shows a schematic diagram of an exemplary system configuration for automated data extraction and analysis of FDA 505(b)(2) applications to generate summaries and/or reports using automated computer processing and/or natural language processing according to some embodiments. System configuration 100 may be used for generating trends, summarizations, and/or reports via an NLP model. Various components of FIG. 1 may be implemented in and/or processed by a processor (e.g., a central processing unit (CPU)) and/or on any number of distributed processors (e.g., a distributed computing system) coupled with memory and connected via a communications network. Each of the components shown in FIG. 1 are described in the context of an exemplary embodiment.

As shown in FIG. 1, embodiments relate to a system configuration 100 for automated data extraction and analysis of FDA 505(b)(2) applications to generate summaries and/or reports using automated computer processing and/or natural language processing. In some embodiments, system configuration 100 may provide graphical user interface (GUI) displays to one or more local or remote display devices. The GUI displays may include one or more data visualization objects related to data associated with new drug applications. In some embodiments, system configuration 100 may include drug application analysis system 102, computing device 104, processor 106, memory 108, display device 110, data files 114-1 through 114-n (referred to individually as data file 114 and collectively as plural data files 114 where appropriate), NLP model 116, and FDA server 118.

In some embodiments, system configuration 100 may operate in a cloud-based environment, such that computing device 104 operates as a remote server in communication with one or more client devices. In some embodiments, system configuration 100 may operate as a distributed system environment, such that a first computing device may collect data associated with new drug applications from an FDA server and the first computing device may store the data in a remote database device and/or on a remote server for access by one or more client devices. For example, computing device 104 may include a server capable of generating an output (e.g., a summary, a trend analysis, a display visualization object, and/or the like) and transmitting the output to the one or more client devices and/or a display device (e.g., one or more display devices) in communication with the one or more client devices such that the output can be processed by the client devices and/or viewed on display devices connected to the client devices. For example, drug application analysis system 102 may be configured as software as a service (SaaS) operating over the Internet to transmit trends, summaries, analyses, and/or display visualization objects to one or more client devices.

In some embodiments, system configuration 100 may include at least one display device (e.g., display device 110). The at least one display device may display GUI displays including at least one data visualization object and/or data associated with new drug applications in a graphical format (e.g., as a graph, histogram, and/or the like). The at least one data visualization object may include a visual object rendered to a display device that represents (e.g., is rendered based on) data associated with new drug applications.

In some embodiments, system configuration 100 may include at least one processor (e.g., processor 106). The at least one processor may be in communication with the at least one display device (e.g., to transmit data and/or display visualization objects for display on the display device via a GUI that is executed and written to the display device by the processor 106). The at least one processor may execute program code for at least one NLP model (e.g., NLP model 116, a trained NLP model, and/or the like). In some embodiments, processor 106 may execute an NLP model and other software instructions concurrently to analyze data associated with new drug applications (e.g., including data in a text format and/or a natural language format).

In some embodiments, system configuration 100 may include memory (e.g., memory 108) storing program instructions including at least one NLP model (e.g., NLP model 116). For example, memory 108 may be in communication with processor 106 and may store program instructions for processor 106 to load and execute to perform functions and/or methods disclosed herein using NLP model 116.

In some embodiments, program instructions stored on memory 108 may cause processor 106 to retrieve plural data files from a server associated with the Food and Drug Administration (FDA), wherein the plural data files include data associated with new drug applications. In some embodiments, the data associated with new drug applications may include data associated with Section 505(b)(2) applications. In some embodiments, the program instructions stored on memory 108 may cause processor 106 to determine that at least one data file includes data for an approved 505(b)(2) drug application.

In some embodiments, when determining that at least one data file includes data for an approved 505(b)(2) drug application, processor 106 may be programmed or configured to extract the data associated with new drug applications from each data file of the plural data files to generate extracted text data for each data file. In some embodiments, processor 106 may identify a first text string associated with a 505(b)(2) drug application in the extracted text data for at least one data file. In some embodiments, processor 106 may identify a second text string associated with a 505(b)(2) drug application in the extracted text data for the at least one data file. The second text string may include at least one numerical character. In some embodiments, the first text string may include “505(b)(2)” and the second text string may include at least “NDA” or “BLA” (in some instances, along with at least one numerical character).

In some embodiments, processor 106 may determine that the at least one data file includes data for an approved 505(b)(2) drug application based on the extracted text data for the at least one data file including the first text string and the second text string. In some embodiments, another text string (e.g., a third text string) may include “indications”, “indications and usage”, and/or “usage” such that processor 106 may perform a text match with text data in at least one data file including data associated with new drug applications. One or more text strings may be used to select and/or generate filters for the text data, such that filters may be used to determine the data associated with new drug applications that may be included in an output and/or an analysis (e.g., summaries, trend analyses, display visualization objects, and/or the like).

In some embodiments, a text string may include textual representations of other aspects of a data file of a NDA (e.g., a warning; a name of a clinical trial or details of a clinical trial, data related to non-clinical trials conducted, a dosage and/or administration of a drug in a data file of an NDA, etc.).

In some embodiments, when processor 106 executes the program instructions, processor 106 may be programmed or configured to extract the data associated with new drug applications from the plural data files as extracted data. In some embodiments, when processor 106 executes the program instructions, processor 106 may be programmed or configured to generate a trend report of new drug applications based on the extracted data and the plural categories. Processor 106 may organize the extracted data into plural categories. The plural categories may be associated with characteristics of new drug applications. For example, at least one of the plural categories may include indications and/or usages of a drug submitted in an NDA represented in the data associated with new drug applications. Processor 106 may detect an indication and/or usage category in the data associated with new drug applications based on performing a text recognition and/or text match operation. Processor 106 may use the indication and/or usage category to detect indication and/or usage data using NLP model 116. Processor 106 may then generate at least one of the plural categories for indications and/or usage as a category for characteristics of new drug applications. Processor 106 may associate (e.g., in a database) the indication and/or usage data with the category for characteristics of new drug applications such that indications and usages may be separately analyzed and/or displayed for various new drug applications.

In some embodiments, processor 106 may extract the indication from a second data file of the plural data files. For example, processor 106 may extract the indication from a data file associated with the at least one data file including data for an approved 505(b)(2) drug application. Where the at least one data file including data for an approved 505(b)(2) drug application is an approved drug application, the data can include an identifier number (e.g., a New Drug Application (NDA) number). The identifier number can be included in the data for other data files, such as a label data file. The label data file can contain the indication and/or usage related to a drug in an approved 505(b)(2) drug application. In this way, the identifier number can associate various data files such that more data can extracted or retrieved. For example, the identifier number can associate an approved 505(b)(2) drug application data file and a label data file, such that additional data can be obtained from the label file (e.g., indication and/or usage data for a drug) that can be included in the text summary. Additionally, indication data can be retrieved by processor 106 from other data files (e.g., additional data files not included in the plural data files) based on identifying the other data files using the identifier number.

In some embodiments, processor 106 may be programmed or configured to determine that the at least one numerical character in the second text string is unique among the plural data files. Processor 106 may be programmed or configured to select the at least one data file to include in the subset of data files based on identifying the first text string associated with a 505(b)(2) drug application in the text data for the at least one data file. Processor 106 may be programmed or configured to identify a file name for the at least one data file. Processor 106 may be programmed or configured to append the second text string and the file name to the list of data files including a subset of plural data files based on determining that the at least one numerical character in the second text string is unique and based on selecting the at least one data file, wherein the second text string and the file name are associated in the list of data files.

In some embodiments, processor 106 may be programmed or configured to generate a flag for the at least one data file including data for an approved 505(b)(2) drug application. For example, processor 106 may generate a flag for the at least one data file based on determining that the at least one data file includes data for an approved 505(b)(2) drug application. In some embodiments, processor 106 may forgo generating a flag or processor 106 may generate a negative flag (e.g., 0, “N”, “No”, and/or the like) based on determining that the at least one data file does not include data for an approved 505(b)(2) drug application.

In some embodiments, processor 106 may be programmed or configured to identify a file name for the at least one data file including data for an approved 505(b)(2) drug application. Processor 106 may be programmed or configured to store the flag and the file name for the at least one data file 505(b)(2) including data for an approved 505(b)(2) drug application in the at least one database device, wherein the flag is associated with the file name as stored in the at least one database device.

In some embodiments, the program instructions stored on memory 108 may cause processor 106 to generate a list of data files including a subset of plural data files. Each data file in the subset of plural data files may represent an NDA or a BLA. In some embodiments, the program instructions stored on memory 108 may cause processor 106 to input the subset of plural data files including data for approved 505(b)(2) drug applications into at least one NLP model. In some embodiments, the program instructions stored on memory 108 may cause processor 106 to generate, with the at least one NLP model, a text summary of each data file of the subset of plural data files.

In some embodiments, when inputting the subset of plural data files including data for approved 505(b)(2) drug applications into at least one NLP model, processor 106 may be programmed or configured to input text data (e.g., extracted text data) of the subset of plural data files into the at least one NLP model. The text data may be labeled and/or delimited such that processor 106 may determine which data file of the subset of plural data files the text data belongs to and/or was extracted an. Such a label and/or delimiter may be defined to be associated with the text data, such as in a database, or may be appended to the text data using a data structure (e.g., an associative array, lists and/or sets combined with dictionaries, and/or the like).

In some embodiments, when processor 106 executes the program instructions, processor 106 may be programmed or configured to display a trend analysis of data for approved 505(b)(2) drug applications based on the subset of data files. For example, processor 106 may cause display device 110 to display a GUI including a trend analysis in the form of a histogram displaying approved 505(b)(2) drug applications and/or indications and/or usages of various drugs and/or products described in the approved 505(b)(2) drug applications.

In some embodiments, when processor 106 executes the program instructions, processor 106 may be programmed or configured to display one or more visual indicators representing each data file in the subset of plural data files via display device 110. The one or more visual indicators may be displayed differently based on a number of data files in the list of data files (e.g., a histogram) or a type of data file in the list of data files (e.g., a NDA, a BLA, or an irrelevant data file).

In some embodiments, system configuration 100 may include a database device (e.g., database device 112). The database device may store data associated with new drug applications (e.g., raw data and/or extracted data). The database device may include a computing device having a database (e.g., executing database software) and configured to store and retrieve data from the database. In some embodiments, the database device may include a database server. The database device may be in communication with (e.g., accessible by) one or more client devices. In some embodiments, the database device may store one or more data files including data associated with new drug applications. The database device may store data associated with new drug applications in various data formats.

In some embodiments, processor 106 may be programmed or configured (e.g., via software instructions) to cause the processor to execute at least one NLP model (e.g., NLP model 116). The at least one NLP model executed by processor 106 may analyze text data and may process natural language in the text data to detect new drug applications (e.g., 505(b)(2) applications) and/or to generate outputs including summaries of new drug applications represented in a natural language output that may be displayed and/or rendered on at least one display device.

In some embodiments, processor 106 may be programmed or configured to cause the processor to input data associated with new drug applications into at least one NLP model (e.g., NLP model 116) executed by processor 106. For example, the processor may input the data associated with new drug applications extracted from one or more data files into an NLP model to determine that at least one the data files is a new drug application (e.g., a 505(b)(2) application), generate an output such as a summary of a data file, or another natural language output based on analysis of the data associated with new drug applications extracted from the one or more data files.

In some embodiments, processor 106 may be programmed or configured to cause the processor to display, via the display device, a GUI displaying at least one display visualization object based on the data associated with new drug applications, such as a textual summary of a new drug application, a graph or histogram of new drug applications, and/or the like. In some embodiments, the NLP model may generate at least one output based on the data associated with new drug applications. In some embodiments, the at least one output may include text data, a prediction about input text data, a numerical value, or a display visualization object displayed in the GUI representing one or more numerical values. In some embodiments, the at least one output may include text representing words and/or phrases generated by the NLP model.

As shown in FIG. 1, drug application analysis system 102 may include software instructions (e.g., program code) implemented on computing device 104. Drug application analysis system 102 may include memory 108 storing the software instructions. Drug application analysis system 102 may include processor 106 executing the software instructions to cause processor 106 to perform one or more functions of drug application analysis system 102. Drug application analysis system 102 may include database device 112. In some embodiments, database device 112 may be a component of (e.g., part of) drug application analysis system 102. Alternatively, database device 112 may be separate from (e.g., as a remote device) drug application analysis system 102. Drug application analysis system 102 may be in communication with at least one server, such as FDA server 118. In some embodiments, database device 112 may be integrated with (e.g., a component of) at least one server. In some embodiments, processor 106 may be integrated with (e.g., a component of) at least one client device.

In some embodiments, when processor 106 retrieves the plural data files from a server associated with the FDA, processor 106 may be programmed or configured to download the plural data files from the server associated with the FDA based on a uniform resource locator (URL) for the plural data files stored on the server associated with the FDA. Processor 106 may store the plural data files in an output folder residing on (e.g., stored in) memory of a server and/or memory of at least one client device.

Drug application analysis system 102 may include at least one NLP model that may be trained (e.g., NLP model 116). At least one NLP model may generate at least one output based on at least data associated with new drug applications received from a client device being provided as a runtime input to at least one NLP model. An output of at least one NLP model may include a textual output, an entity determination (e.g., determination that a data file is a new drug application), one or more lists of topics (e.g., topic modeling), and/or a textual summary.

In some embodiments, drug application analysis system 102 may be implemented in a single computing device. In some embodiments, drug application analysis system 102 may be implemented in plural computing devices (e.g., a group of servers, such as a group of computing devices 104, and/or the like) as a distributed system such that software instructions and/or NLP models are implemented on different computing devices. In some embodiments, drug application analysis system 102 may be associated with computing device 104, such that drug application analysis system 102 is executed on computing device 104 or a portion of drug application analysis system 102 is executed on computing device 104 as part of a distributed computing system where database device 112 is not a component of computing device 104 and/or drug application analysis system 102. Alternatively, drug application analysis system 102 may include at least one computing device 104 executing software instructions and at least one database device 112.

Computing device 104 may include one or more processors (e.g., processor 106) configured to execute software instructions. For example, computing device 104 may include a desktop computer, a portable computer (e.g., laptop computer, tablet computer), a workstation, a mobile device (e.g., smartphone, cellular phone, personal digital assistant, wearable device), a server, and/or other like devices. Computing device 104 may include a computing device configured to communicate with one or more other computing devices over a network. Computing device 104 may include a group of computing devices (e.g., a group of servers) and/or other like devices. In some embodiments, computing device 104 may include a data storage device. Alternatively, a data storage device may be separate from computing device 104 and may be in communication with computing device 104 (e.g., database device 112). Computing device 104 may include processor 106 (e.g., CPU) and memory 108. Processor 106 may execute software instructions (e.g., compiled program code) for drug application analysis system 102, including software instructions for at least one NLP model (e.g., a trained NLP model).

Processor 106 may be implemented in hardware, software, or a combination of hardware and software. For example, processor 106 may include a common processor (e.g., CPU), a graphics processing unit (GPU), an accelerated processing unit (APU), a microprocessor, a digital signal processor (DSP), and/or any processing component (e.g., a field-programmable gate array (FPGA), an application-specific integrated circuit (ASIC), etc.) that can be programmed with software instructions such that the processor is configured to cause the processor to perform functions when executing the software instructions. In some embodiments, processor 106 may include plural processors implemented in a single computing device 104 (e.g., a CPU and a GPU) or processor 106 may include plural processors implemented among plural distributed computing devices 104. Processor 106 may be coupled to memory 108 via a data bus to transfer data between processor 106 and memory 108. In some embodiments, processor 106 may be coupled to display device 110 via wired (e.g., a data bus, ethernet, and/or the like) or wireless (e.g., Wi-Fi, Bluetooth, and/or the like) means and/or a communication interface.

Memory 108 may include random access memory (RAM), read-only memory (ROM), and/or another type of dynamic or static storage device (e.g., flash memory, magnetic memory, optical memory, etc.) that stores information and/or software instructions for use by processor 106. Memory 108 may include a computer-readable medium and/or a storage component. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 108 from another computer-readable medium or from another device via a communication interface with computing device 104. When executed, software instructions stored in memory 108 and executed by processor 106 may cause processor 106 to perform one or more functions described herein. Data files 114 shown in FIG. 1 may include electronic files including textual data and/or image data (e.g., PDF files, text files, image files, and/or the like) such that a data file 114 may include independent data (e.g., data associated with new drug applications) that may not be included in other data files 114. For example, each PDF file of plural PDF files may include a set of data associated with new drug applications, where at least a portion of the data in each PDF file may be unique. In another example, each text file of plural text files may include a set of data associated with new drug applications, where at least a portion of the data in each text file may be unique. In some embodiments, each text file of plural text files may include data associated with plural new drug applications, such that each text file includes metadata associated with plural new drug applications (e.g., application number, application URL, and/or the like). In some embodiments, data files 114 may include different types of data files (e.g., such that data file 114-1 is a text file and data file 114-2 is a PDF file, etc.).

Display device 110 may include a video display such as a liquid crystal display (LCD) or a light-emitting diode (LED) display. Display device 110 may include, for example, a television, a computer monitor, a head-mounted display or “heads-up” display, a virtual reality headset, a mobile display on a mobile device and/or smartphone, a projector, or any other display device that is configured to display computer generated images. Display device 110 may be in communication with computing device 104 and/or processor 106 such that processor 106 may transmit and/or write data to display device 110 to cause display device 110 to display images such as a GUI including at least one display visualization object. In some embodiments, display device 110 may be in communication with a client device and computing device 104 and/or processor 106 may transmit and/or write data to the client device which then can transmit and/or write the data to display device 110. Display device 110 may be in communication with computing device 104 and/or processor 106 via wired (e.g., a data bus, ethernet, and/or the like) or wireless (e.g., Wi-Fi, Bluetooth, and/or the like) means and/or a communication interface. In some embodiments, display device 110 may be separate from computing device 104. Alternatively, display device 110 may be integrated with (e.g., part of) computing device 104, for example, where computing device 104 includes a mobile device such as a smartphone.

Database device 112 may include a computing device having a database (e.g., executing database software) and configured to store and retrieve data from the database. In some embodiments, the database device may include one or more database servers, such as in a data warehouse and/or data lake. Database device 112 may be in communication with (e.g., accessible by) one or more client devices. In some embodiments, database device 112 may store one or more data files (e.g., data files 114) and/or data associated with new drug application. Database device 112 may include a database using a key-value format, such as a non-relational database (e.g., a NoSQL database).

NLP model 116 may include at least one NLP model, such as a trained text summarization model, a text classification model, a probabilistic language model, a feature extraction model, a keyword extraction model, or another type of NLP model. NLP model 116 may be trained based on a natural language processing algorithm and/or a machine learning algorithm, such as logistic regression, decision trees, random forest, naïve bayes, support vector machines, artificial neural networks, or other types of machine learning algorithms. At least one NLP model 116 may generate a first output (e.g., a text summary, an entity recognition, a keyword, and/or the like) based on a runtime input provided to the at least one NLP model 116 (e.g., a trained NLP model 116). In some embodiments, drug application analysis system 102 (e.g., via processor 106) may execute a plural NLP models 116 concurrently.

FDA server 118 may include a computing device associated with one or more government agencies (e.g., the server may be physically located at a headquarters of a government agency and/or the server may be controlled by one or more government agencies). In some embodiments, FDA server 118 may include a plurality of servers and/or a plurality of computing devices. FDA server 118 may communicate with drug application analysis system 102 (e.g., via computing device 104) and/or one or more client devices via a communication network.

The number and arrangement of systems, hardware, and/or modules (e.g., software modules, software instructions) shown in FIG. 1 is provided as an example. There may be additional systems, hardware, and/or modules, fewer systems, hardware, and/or modules, different systems, hardware, and/or modules, or differently arranged systems, hardware, and/or modules than those shown in FIG. 1. Furthermore, two or more systems, hardware, and/or modules shown in FIG. 1 may be implemented within a single system, hardware, and/or module. A single system, hardware, and/or module shown in FIG. 1 may be implemented as multiple, distributed systems, hardware, and/or modules. For example, a first software module may be executed on a first computing device where a second software module is executed on a second computing device that is remote (e.g., off-site, located in a separate building or structure, a substantial distance away, and/or the like) from the first computing device. Additionally or alternatively, a set of systems, a set of hardware, and/or a set of modules (e.g., one or more systems, one or more hardware devices, one or more modules) of FIG. 1 may perform one or more functions described as being performed by another set of systems, another set of hardware, or another set of modules of FIG. 1.

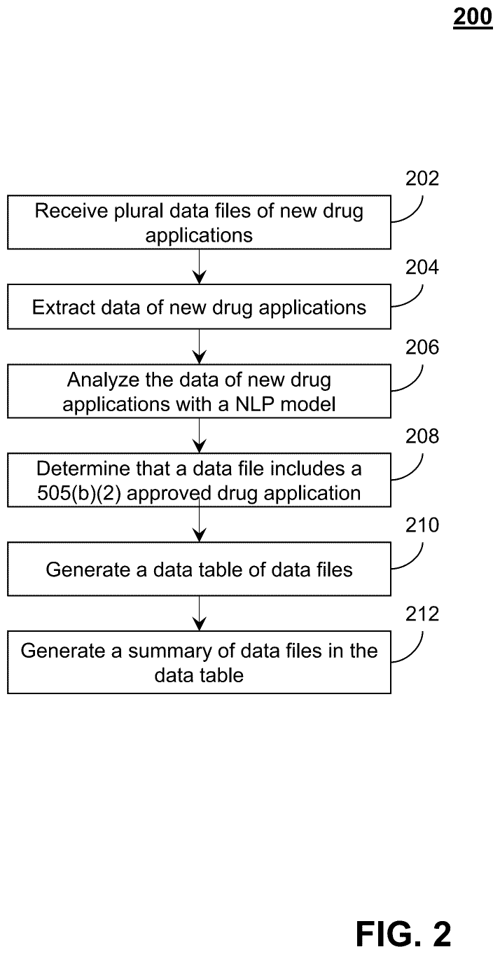

Referring now to FIG. 2, shown is a flow diagram of a method 200 for automated data extraction and analysis of FDA 505(b)(2) applications to generate summaries and/or reports using automated computer processing and/or natural language processing according to some embodiments. The steps shown in FIG. 2 are for example purposes only. It will be appreciated that additional, fewer, different, and/or a different order of steps may be used in some embodiments.

At step 202, method 200 may include receiving plural data files of new drug applications. For example, method 200 may include receiving plural data files from a server. The plural data files may include data representing new drug applications submitted to a government agency (e.g., the FDA). In some embodiments, the plural data files may include plural PDF files including text data and/or image data. Additionally or alternatively, the plural data files may include plural text files including text data. The plural data files may be stored on a server associated with a government agency, such as the FDA (e.g., FDA server 118). The data representing new drug applications may have been received electronically by the government agency from one or more client devices (e.g., as user input). Additionally or alternatively, the data representing new drug applications may have been submitted to the government agency manually (e.g., not electronically) via mail or another method such that the government agency (e.g., personnel of the government agency, users, and/or the like) may have entered the data representing new drug applications into a computing device to store the data representing new drug applications electronically. In some embodiments, the data representing new drug applications may include data associated with section 505(b)(2) applications.

In some embodiments, processor 106 may receive plural data files of various other applications. For example, processor 106 may receive data files including data representing a medical device 510(k) application, a premarket approval (PMA) application, a BLA, and/or other types of FDA application pathways. It is to be understood that the systems and methods provided herein are not limited to data files for NDAs, and systems and methods described herein may be used with data files for other application types and/or data types accessible via a server (e.g., an FDA server).

At step 204, method 200 may include extracting data of new drug applications. For example, method 200 may include extracting the data representing new drug applications from the plural data files to generate extracted text data. Processor 106 may extract the data associated with new drug applications from the plural data files based on performing text recognition on text data in the plural data files, performing optical character recognition (OCR) on image data in the plural data files, and/or inputting the text data in the plural data files into an NLP model. In some embodiments, processor 106 may store the extracted text data in at least one database (e.g., database device 112). For example, processor 106 may perform OCR on image data in at least one data file of the plural data files. The image data may include images of printed text, images of handwritten text, and/or other image data.

In some embodiments, processor 106 may perform a first extraction of data representing new drug applications from plural text files to generate text data including new drug application numbers, new drug application type identifiers (IDs), new drug application types, and/or other data related to new drug applications. Processor 106 may identify one or more PDF files including data representing new drug applications for downloading based on analyzing the text data to determine which PDF files of new drug applications were not previously downloaded and/or analyzed (e.g., based on an application number). Processor 106 may then download the identified one or more PDF files from each URL of the one or more PDF files, each URL obtained from the text data having been extracted from the plural text files. Once the one or more PDF files including data associated with new drug applications have been downloaded and stored (e.g., to memory 108 residing on computing device 104), processor 106 may perform a second extraction of data representing new drug applications from the one or more PDF files to generate the extracted text data. In this way, embodiments may provide for more targeted downloaded of data files including data representing new drug applications by first analyzing text data from plural text files (e.g., metadata associated with new drug applications) and extracting text data from PDF files that have not been previously downloaded or have had data representing new drug applications extracted from the files. This may also provide a method for updating a dataset to include data representing new drug applications by only extracting and analyzing extracted data from PDF files which have not been previously downloaded, thus forgoing any PDF files that have been previously downloaded and have had data extracted and analyzed.

At step 206, method 200 may include analyzing the data of new drug applications with an NLP model. For example, method 200 may include analyzing the extracted text data using an NLP model. Processor 106 may input the extracted text data into at least one NLP model for processing.

At step 208, method 200 may include determining that a data file includes an approved drug application. For example, method 200 may include determining that at least one data file includes data for an approved 505(b)(2) drug application based on analyzing the text data using the NLP model. In some embodiments, processor 106 may determine that at least one data file includes data for an approved 505(b)(2) drug application based on extracting the data associated with new drug applications from the plural data files and performing text recognition on the data associated with new drug applications.

At step 210, method 200 may include generating a data table of data files. For example, method 200 may include generating a data table based on analyzing the extracted text data. The data table may include one or more data files of the plural data files that were determined to include data for an approved 505(b)(2) drug application. Each data file of the one or more data files may be associated with a flag (e.g., a Boolean flag) in the data table. In some embodiments, the data table may include a table stored in a database, a spreadsheet, and/or other electronic table format. In some embodiments, the flag associated with each data file may indicate whether the data file was determined to include data for an approved 505(b)(2) drug application. For example, a first data file may be associated with a positive flag (e.g., 1, “Y”, “Yes”, and/or the like) where the data file was determined to include data for an approved 505(b)(2) drug application. A second data file may be associated with a negative flag (e.g., 0, “N”, “No”, and/or the like) where the data file was determined to not include data for an approved 505(b)(2) drug application.

At step 212, method 200 may include generating a summary of data files in the data table. For example, method 200 may include generating, with the at least one NLP model, a summary of the data for an approved 505(b)(2) drug application in each data file of the one or more data files in the data table. In some embodiments, the summary may include a textual summary of the data for an approved 505(b)(2) drug application extracted from at least one data file of the plural data files. In some embodiments, processor 106 may generate a summary of the data for an approved 505(b)(2) drug application for data files that are associated a positive flag in the data table and not for data files that are associated with a negative flag in the data table.

In some embodiments, processor 106 may organize the extracted text data into plural categories. The plural categories may be associated with characteristics of new drug applications (e.g., names of drugs, indications and/or usages of drugs, and/or the like). In some embodiments, processor 106 may generate a trend report of new drug applications based on the extracted text data and the plural categories. For example, processor 106 may generate the trend report (e.g., by writing data for the trend report to a display device) such that the trend report includes display visualization objects (e.g., on a GUI via a display device) for each of the plural categories having varying sizes based on the extracted text data.

In some embodiments, processor 106 may combine and/or associate the extracted text data representing new drug applications with economic data (or another type of data) obtained from a separate database and/or server. For example, processor 106 may associate data representing a new drug application with sales data of the new drug application obtained from a database controlled by a manufacturer of a drug named in the new drug application. The economic data (or another type of data) may be provided (e.g., combined) in a display and/or summary along with the extracted text data. The economic data (or another type of data) may be associated in a database with the data representing new drug applications.

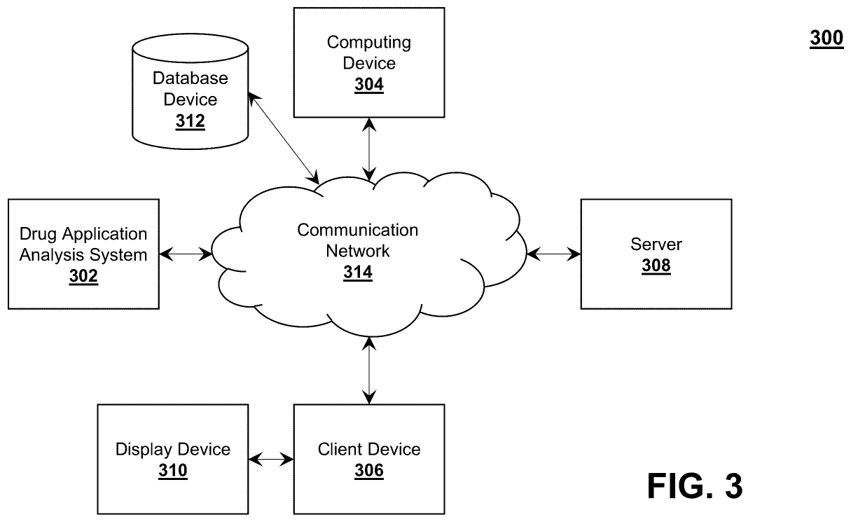

FIG. 3 shows a diagram of an embodiment of an exemplary environment 300 in which systems, products, and/or methods, as described herein, may be implemented. As shown in FIG. 3, environment 300 may include drug application analysis system 302, computing device 304, client device 306, server 308, display device 310, database device 312, and communication network 314. In some embodiments, each of computing device 304, client device 306, server 308, display device 310, database device 312, and/or communication network 314 may be implemented by (e.g., part of) drug application analysis system 302. In some embodiments, at least one of each of computing device 304, client device 306, server 308, display device 310, database device 312, and/or communication network 314 may be implemented by (e.g., part of) another system, another device, another group of systems, or another group of devices, separate from or including drug application analysis system 302, such as computing device 304, client device 306, server 308, and/or the like.

Drug application analysis system 302 may include one or more devices capable of receiving information from and/or communicating information to computing device 304, client devices 306, server 308, and/or database device 312 via communication network 314. For example, drug application analysis system 302 may include a computing device, such as a server, a group of servers, and/or other like devices. In some embodiments, drug application analysis system 302 may be associated with a server as described herein. In some embodiments, drug application analysis system 302 may be in communication with a data storage device (e.g., database, memory, database device 312, and/or the like), which may be local or remote to drug application analysis system 302. In some embodiments, drug application analysis system 302 may be capable of receiving information from, storing information in, communicating information to, or searching information stored in the data storage device.

Computing device 304 may include one or more devices capable of receiving information and/or communicating information to drug application analysis system 302, client device 306, server 308, and/or database device 312 via communication network 314. For example, computing device 304 may include a computing device, such as a server, a group of servers, and/or other like devices. In some embodiments, computing device 304 may be associated with a server, a client device, and/or a user device as described herein.

Client device 306 may include one or more devices capable of receiving information from and/or communicating information to drug application analysis system 302, computing device 304, and/or server 308 via communication network 314. In some embodiments, client device 306 may be prohibited and/or blocked from communication with database device 312. Client device 306 may transmit data to database device 312 only through drug application analysis system 302, computing device 304, and/or server 308. Additionally or alternatively, one or more client devices 306 may include a device capable of receiving information from and/or communicating information to other client devices 306 via communication network 314, another network (e.g., an ad hoc network, a local network, a private network, a virtual private network, and/or the like), and/or any other suitable communication technique. For example, client device 306 may include a user device and/or the like. Client device 306 may also be in communication with a local or remote display device 310.

Server 308 may include a computing device. In some embodiments, server 308 may include a plurality of servers and/or a plurality of computing devices. Server 308 may communicate with computing device 304 and/or client device 306 via communication network 314.

Display device 310 may include a video display such as a liquid crystal display (LCD) or a light-emitting diode (LED) display. Display device 310 may include, for example, a television, a computer monitor, a head-mounted display or “heads-up” display, a virtual reality headset, a mobile display on a mobile device and/or smartphone, a projector, or any other display device that is configured to display computer generated images. Display device 310 may be in communication with computing device 304 and/or client device 306 such that client device 306 may transmit and/or write data to display device 310 to cause display device 310 to display images such as a GUI. In some embodiments, display device 310 may be in communication with client device 306 and/or computing device 304 such that computing device 304 may transmit and/or write data to client device 306 which may then transmit and/or write the data to display device 310. Display device 310 may be in communication with computing device 304 and/or client device 306 via wired (e.g., a data bus, ethernet, and/or the like) or wireless (e.g., Wi-Fi, Bluetooth, and/or the like) means and/or a communication interface. In some embodiments, display device 310 may be separate from computing device 304 and/or client device 306. Alternatively, display device 310 may be integrated with (e.g., part of) computing device 304 and/or client device 306, for example, where computing device 304 and/or client device 306 include a mobile device such as a smartphone. In some embodiments, computing device 304 and client device 306 may include a same device and/or computing device 304 and client device 306 may share a same display device 310.

Communication network 314 may include one or more wired and/or wireless networks. For example, communication network 314 may include a cellular network (e.g., a long-term evolution (LTE®) network, a third generation (3G) network, a fourth generation (4G) network, a fifth generation (5G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network (e.g., a private network associated with drug application analysis system 302), an ad hoc network, an intranet, the Internet, a fiber optic-based network, a cloud computing network, and/or the like, and/or a combination of these or other types of networks.

The number and arrangement of systems, devices, and/or networks shown in FIG. 3 are provided as an example. There may be additional systems, devices, and/or networks; fewer systems, devices, and/or networks; different systems, devices, and/or networks; and/or differently arranged systems, devices, and/or networks than those shown in FIG. 3. Furthermore, two or more systems or devices shown in FIG. 3 may be implemented within a single system or device, or a single system or device shown in FIG. 3 may be implemented as multiple, distributed systems or devices. Additionally or alternatively, a set of systems (e.g., one or more systems) or a set of devices (e.g., one or more devices) of environment 300 may perform one or more functions described as being performed by another set of systems or another set of devices of environment 300.

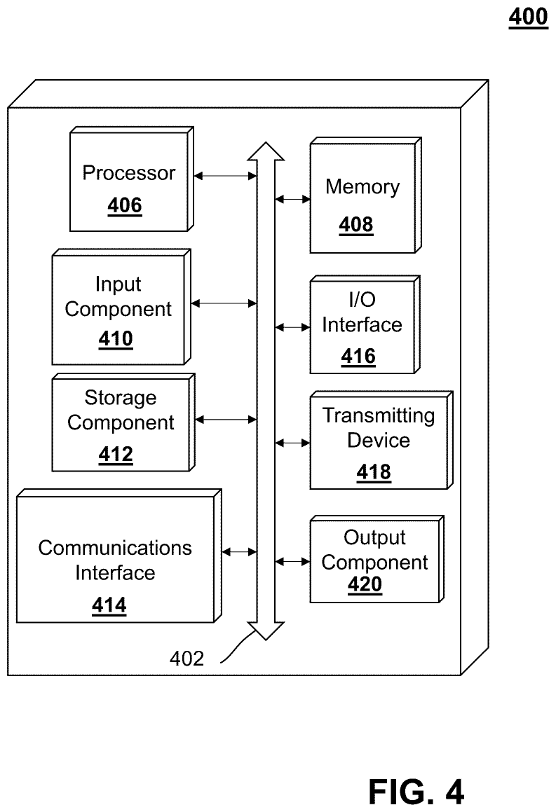

FIG. 4 shows a diagram of example components of a device 400 according to some embodiments. Device 400 (and/or at least one component of device 400) may correspond to at least one of drug application analysis system 102, computing device 104, display device 110, and/or database device 112 in FIG. 1 and/or at least one of drug application analysis system 302, computing device 304, client device 306, server 308, display device 310, and/or database device 312 in FIG. 3, as an example. In some embodiments, such systems or devices in FIG. 1 or FIG. 3 may include at least one device 400 and/or at least one component of device 400. The number and arrangement of components shown in FIG. 4 are provided as an example. In some embodiments, device 400 may include additional components, fewer components, different components, or differently arranged components than those shown in FIG. 4. Additionally or alternatively, a set of components (e.g., one or more components) of device 400 may perform one or more functions described as being performed by another set of components of device 400.

As shown in FIG. 4, device 400 may include bus 402, processor 406, memory 408, input component 410, storage component 412, communication interface 414, input/output (I/O) interface 416, transmitting device 418, and output component 420. Bus 402 may include a component that permits communication among the components of device 400. In some embodiments, processor 406 may be implemented in hardware, software (e.g., firmware), or a combination of hardware and software. For example, processor 406 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 408 may include RAM, ROM, and/or another type of dynamic or static storage device (e.g., flash memory, magnetic memory, optical memory, etc.) that stores information and/or instructions for use by processor 406. In some embodiments, processor 406 may be the same as or similar to processor 106. In some embodiments, memory 408 may be the same as or similar to memory 108.

Input component 410 may include a component that permits device 400 to receive information, such as via user input (e.g., a touch screen display, a keyboard, a keypad, a mouse, a button, a switch, a microphone, etc.). Additionally or alternatively, input component 410 may include a sensor for sensing information (e.g., a global positioning system (GPS) component, an accelerometer, a gyroscope, an actuator, etc.).

Storage component 412 may store information and/or software related to the operation and use of device 400. For example, storage component 412 may include a hard disk (e.g., a magnetic disk, an optical disk, a magneto-optic disk, a solid state disk, etc.) and/or another type of computer-readable medium. In some embodiments, storage component 412 may be the same as or similar to database device 112 and/or database device 312.

Communication interface 414 may permit device 400 to receive information from another device and/or provide information to another device. For example, communication interface 414 may include an Ethernet interface, an optical interface, a coaxial interface, an infrared interface, a radio frequency (RF) interface, a universal serial bus (USB) interface, a Wi-Fi® interface, a cellular network interface, and/or the like. In some embodiments, communications interface may be the same as or similar to communication network 314.

I/O interface 416 may be configured to receive signals from processor 406 and generate an output suitable for a peripheral device via a direct wired or wireless link. I/O interface 416 may include a combination of hardware and software for example, a processor, circuit card, or any other suitable hardware device encoded with program code, software, and/or firmware for communicating with a peripheral device such as a display device, printer, audio output device, or other suitable electronic device or output type as desired.

Transmitting device 418 may be configured to receive data from processor 406 and may be configured to assemble the data into a data signal and/or data packets according to the specified communication protocol and data format of a peripheral device or remote device to which the data is to be sent. Transmitting device 418 may include any one or more of hardware and software components for generating and communicating the data signal over communications interface 414 and/or via a direct wired or wireless link to a peripheral or remote device. Transmitting device 418 may be configured to transmit information according to one or more communication protocols and data formats. In some embodiments, transmitting device 418 may include or may be coupled with a receiving device (e.g., a transceiver, and/or the like).

Output component 420 may include a component that provides output information from device 400 (e.g., a display, a speaker, one or more light-emitting diodes (LEDs), etc.). Communication interface 414 may include a transceiver-like component (e.g., a transceiver, a separate receiver and transmitter, etc.) that enables device 400 to communicate with other devices, such as via a wired connection, a wireless connection, or a combination of wired and wireless connections. In some embodiments, output component 420 may be the same as or similar to display device 110 and/or display device 310.

Device 400 may perform one or more processes described herein. Device 400 may perform these processes based on processor 406 executing software instructions stored by a computer-readable medium, such as memory 408 and/or storage component 412. A computer-readable medium may include any non-transitory memory device. A memory device includes memory space located inside of a single physical storage device or memory space spread across multiple physical storage devices. Software instructions (e.g., software modules) may be read into memory 408 and/or storage component 412 from another computer-readable medium or from another device via communication interface 414. When executed, software instructions stored in memory 408 and/or storage component 412 may cause processor 406 to perform one or more processes described herein. Additionally or alternatively, hardwired circuitry may be used in combination with software instructions to perform one or more processes described herein. Thus, embodiments described herein are not limited to any specific combination of hardware circuitry and software. The term “programmed or configured,” as used herein, may refer to an arrangement of software, hardware circuitry, or any combination thereof on one or more devices.

Further details regarding embodiments of systems, methods, and computer program products for automated data extraction and analysis of FDA 505(b)(2) applications to generate summaries and/or reports using automated processes and/or natural language processing are disclosed in the Appendix filed herewith, the entire disclosure of which is hereby incorporated by reference in its entirety.

Although embodiments have been described in detail for the purpose of illustration, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A system for automatically generating reports of new drug applications, the system comprising:
memory storing program instructions; and
at least one processor configured to execute the program instructions, wherein when the at least one processor executes the program instructions, the at least one processor will be programmed or configured to:
retrieve plural data files from a server associated with the Food and Drug Administration (FDA), wherein the plural data files include data associated with new drug applications;
determine that at least one data file includes data for an approved 505(b)(2) drug application;
generate a list of data files including a subset of plural data files, wherein each data file in the subset of plural data files represents a new drug application;
input the subset of plural data files including data for approved 505(b)(2) drug applications into at least one natural language processing (NLP) model; and
generate, with the at least one NLP model, a text summary of each data file of the subset of plural data files.

2. The system of claim 1, wherein, when determining that at least one data file includes data for an approved 505(b)(2) drug application, the at least one processor will be programmed or configured to:
extract the data associated with new drug applications from each data file of the plural data files to generate extracted text data for each data file;
identify a first text string associated with a 505(b)(2) drug application in the extracted text data for the at least one data file;
identify a second text string associated with a 505(b)(2) drug application in the extracted text data for the at least one data file, wherein the second text string includes at least one numerical character; and
determine that the at least one data file includes data for an approved 505(b)(2) drug application based on the extracted text data for the at least one data file including the first text string and the second text string.

3. The system of claim 2, wherein, when inputting the subset of plural data files including data for approved 505(b)(2) drug applications into at least one NLP model, the at least one processor will be programmed or configured to:
input text data of the subset of plural data files into the at least one NLP model.

4. The system of claim 2, wherein the first text string includes “505(b)(2)”, and the second text string includes at least “NDA” or “BLA”.

5. The system of claim 2, wherein the first text string includes any one of “indications” and “usage”.

6. The system of claim 2, wherein, when the at least one processor executes the program instructions, the at least one processor will be programmed or configured to:
determine that the at least one numerical character in the second text string is unique among the plural data files;
select the at least one data file to include in the subset of data files based on identifying the first text string associated with a 505(b)(2) drug application in the text data for the at least one data file;
identify a file name for the at least one data file; and
append the second text string and the file name to the list of data files including a subset of plural data files based on determining that the at least one numerical character in the second text string is unique and based on selecting the at least one data file, wherein the second text string and the file name are associated in the list of data files.

7. The system of claim 1 in combination with at least one database device, wherein, when the at least one processor executes the program instructions, the at least one processor will be programmed or configured to:
generate a flag for the at least one data file 505(b)(2) including data for an approved 505(b)(2) drug application;
identify a file name for the at least one data file including data for an approved 505(b)(2) drug application;
store the flag and the file name for the at least one data file 505(b)(2) including data for an approved 505(b)(2) drug application in the at least one database device, wherein the flag is associated with the file name as stored in the at least one database device.

8. The system of claim 1, wherein the data associated with new drug applications includes data associated with Section 505(b)(2) applications.

9. The system of claim 1, wherein, when the at least one processor executes the program instructions, the at least one processor will be programmed or configured to:
display a trend analysis of data for approved 505(b)(2) drug applications based on the subset of data files.

10. The system of claim 1 in combination with at least one display device, wherein, when the at least one processor executes the program instructions, the at least one processor will be programmed or configured to:
display one or more visual indicators representing each data file in the subset of plural data files via the at least one display device, wherein the one or more visual indicators are displayed based on a number of data files in the list of data files.

11. The system of claim 1, wherein when the at least one processor executes the program instructions, the at least one processor will be programmed or configured to:
extract the data associated with new drug applications from the plural data files as extracted data;
organize the extracted data into plural categories, the plural categories being associated with characteristics of new drug applications; and
generate a trend report of new drug applications based on the extracted data and the plural categories.

12. The system of claim 1, wherein the plural data files are portable document format (PDF) files.

13. The system of claim 7, wherein the database device is integrated with at least one server and wherein the at least one processor is integrated with at least one client device.

14. The system of claim 1, wherein, when the at least one processor retrieves the plural data files from a server associated with the Food and Drug Administration (FDA), the at least one processor will be programmed or configured to:
download the plural data files from the server associated with the FDA based on a uniform resource locator (URL) associated with the plural data files stored on the server associated with the FDA; and
store the plural data files in an output folder residing on at least one client device.

15. A method for automatically generating reports for new drug applications, the method comprising:
receiving, with at least one processor, plural data files from a server, the plural data files including data representing new drug applications submitted to a government agency;
extracting, with the at least one processor, the data representing new drug applications from the plural data files to generate extracted text data;
analyzing, with the at least one processor, the extracted text data using a natural language processing (NLP) model;
determining, with the at least one processor, that at least one data file includes data for an approved 505(b)(2) drug application based on analyzing the text data using the NLP model;
generating, with the at least one processor, a data table based on analyzing the text data, the data table including one or more data files of the plural data files that were determined to include data for an approved 505(b)(2) drug application, each data file of the one or more data files associated with a flag in the data table; and
generating, with the at least one NLP model, a summary of the data for an approved 505(b)(2) drug application in each data file of the one or more data files in the data table.

16. The method of claim 15, wherein the data representing new drug applications includes data associated with Section 505(b)(2) applications.

17. The method of claim 15, further comprising:
organizing, with the at least one processor, the text data into plural categories, the plural categories being associated with characteristics of new drug applications; and
generating a trend report of new drug applications based on the text data and the plural categories.
